(19) United States
(12) Patent Application Publication
(10) Pub. No.: US 2005/0020995 A1
(43) Pub. Date: Jan. 27, 2005

(54) Peritoneal dialysis security band and methods of use

(76) Inventor: Sheila Shaw, Tracy, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/690,289

(22) Filed: Oct. 21, 2003

Related U.S. Application Data
(60) Provisional application No. 60/488,958, filed on Jul. 22, 2003.

(51) Int. Cl. A61M 31/00

(52) U.S. Cl. 604/500; 604/179

(57) ABSTRACT

The present invention provides a peritoneal dialysis security band including an elastic band and a catheter retaining pocket positioned along a portion of the elastic band. The elastic band is not detachable and the catheter retaining pocket is able to accept a catheter. The present invention also includes methods of use.

### CROSS REFERENCE TO RELATED APPLICATIONS

The present application claims benefit of priority to Provisional Application No. ______ (to be determined), entitled Peritoneal Dialysis Security Band, filed (date), and naming Sheila Shaw as inventor, which is herein incorporated by reference in its entirety.

### TECHNICAL FIELD

The present invention relates to medical devices, and more particularly relates to a non-detachable peritoneal dialysis security band for retaining the external portion of a peritoneal dialysis catheter when not in use.

### BACKGROUND OF THE INVENTION

Peritoneal dialysis is a technique intended to mimic the effect of the kidneys and involves the removal of soluble waste substances from the body by a transfer process across the peritoneum. This procedure, often referred to as an exchange, includes introducing a dialysis solution into the peritoneum of an individual, allowing the solution to collect soluble waste and draining the waste solution. Fluids are transferred using a surgically implanted device called a catheter. Without proper care, the catheter can become dislodged resulting in infection and potential death.

Since the catheter has an external portion, the patient must store the external portion of the catheter along the body. Traditionally, a patient would adhesively tape the external portion of the catheter to the abdomen. However, this technique can be uncomfortable to the patient and frequent removal and re-taping of the catheter often causes irritation.

Several U.S. patents offer an alternative to taping the catheter to the abdomen. These devices frequently incorporate a detachable belt. Generally the opposing ends of a detachable belt are reversibly fastened using a fastening means or fastening structure. Examples include hooks, snaps, buttons, buckles, VELCRO (R) the like. However, there are multiple disadvantages to a detachable peritoneal dialysis belt. First, a fastening structure may lead to binding, pinching, scratching, or irritation of the skin. Second, the belt is typically fastened after retention of a catheter, which may lead to an uncomfortable pulling or tugging on the catheter while attempting to fasten the ends. Third, since the belt may be reversibly fastened there is a risk of the garment coming undone. Once the garment comes undone, the catheter may be exposed to a risk of becoming dislodged, pulled, or stressed. Fourth, if the fastening structure is constructed from metal, the patient risks exposure to rust after machine washing the device or while the device rests against the moisture of the patient's body.

U.S. Pat. No. 5,468,229 discloses a reversibly securable peritoneal dialysis belt having an aperture for receiving and orientating the protruding portion of a catheter towards a plurality of holders along one portion of the belt. Referring to column 2, line 25, this device requires the exterior portion of the catheter be inserted through an aperture and guided through the plurality of holders thereby securing and supporting the entire external portion of the catheter. Referring to column 3, line 1, the securing means may be a hook and loop, snap or button. A disadvantage of this device is that feeding the catheter through the aperture then through the plurality of holders involves significant manipulation of the catheter. This manipulation may lead to pulling the catheter causing discomfort. Secondly, the catheter must be fed through the aperture and likely through the holders prior to attaching the ends of the belt. Therefore fastening the ends may include pulling or tugging the retained catheter causing discomfort. Thirdly, a fastening structure may lead to binding, pinching, scratching, or irritation of the skin. Fourthly, because the belt may be reversibly fastened, there is a risk of the garment coming undone.

U.S. Pat. No. 6,436,074 discloses a garment for securing and exposing a peritoneal dialysis catheter and catheter exit site including a slip resistant torso belt, a pouch into which the end of the disconnected catheter can be placed, and a block of foam rubber permanently attached inside the belt. Column 3, line 26 describes the belt of the device as consisting of an elastic strap which can be fastened into a torso belt using Velcro® fasteners. Column 3, line 35 describes the pouch as having an opening once at its top along the length of the pouch and again through a slit at the exit site covering. Therefore this device includes a detachable belt and a pocket containing an entry aperture and an exit aperture along the top. Use of this device requires the catheter be fed through two apertures, which may lead to pulling of the catheter and discomfort. In addition, the belt has detachable ends and therefore the fastening structure may lead to binding, pinching, scratching, or irritation of the skin. Also, because the belt may be reversibly fastened, there is a risk of the garment coming undone.

U.S. Pat. No. 5,853,396 discloses a tuck-away belt for peritoneal dialysis patients including a flexible elongated pocket envelope having two panels and a flexible elastic band narrower than the envelope pocket and having a hook and loop type attachment at one end and a D-ring at the opposite end through which the elastic band is pulled through to attach itself. Thus this device includes a flexible envelope attached to a reversibly attachable narrow belt. This device includes a hook and loop fastener. A hook and loop type fastener may lead to binding, pinching, scratching, or irritation of the skin. In addition since a hook and loop type fastener may be reversibly fastened, there is a risk of the garment coming undone.

U.S. Pat. No. 6,126,639 discloses a continuous ambulatory peritoneal dialysis catheter support under garment including a belt of expandable material, a pocket portion, and a means for releasably attaching a first and second end. Referring to column 2, line 30, the patient inserts the catheter into the pocket portion, wraps the ends of the garment around their waist or hips, and attaches the ends. Referring to column 3, line 58, the releasable attachment means or suitable securing means may utilize hook and loop, buttons, snaps, buckles, re-usable tape, and the like. Thus, this dialysis support undergarment includes a detachable belt with a securing means or fastening structure. The securing means may therefore lead to binding, pinching, scratching, or irritation of the skin. Moreover since the securing means is reversibly fastened, there is a risk of the garment coming undone.

Therefore there is a need for a peritoneal dialysis device that reduces binding, pinching, scratching, or irritation of the skin and reduces the risk of the garment coming undone.

### BRIEF SUMMARY OF THE INVENTION

It is the object of the present invention to provide a peritoneal dialysis security band that reduces, minimizes or eliminates the binding, pinching, scratching, or irritation of the skin that can occur with a catheter belt having detachable ends. It also the object of the present invention to provide a peritoneal dialysis security band that reduces, minimizes or eliminates the risk of the garment coming undone.

Specifically, the present invention provides a peritoneal dialysis security band and a catheter retaining pocket located along a portion of the elastic band. The elastic band is not detachable and the catheter retaining pocket is able to accept the external portion of a catheter.

The present invention also provides a method of retaining the external portion of an implanted peritoneal dialysis catheter including placing the peritoneal dialysis security band of the present invention about the waist or hips of an individual and inserting the peritoneal dialysis catheter within the catheter retaining pocket. The method may also include placing the peritoneal dialysis security band about the waist or hips prior to inserting the catheter in the catheter retaining pocket.

### BRIEF DESCRIPTION OF THE DRAWINGS

FIG. 1 depicts an elevated front right view of a peritoneal dialysis security band 10. The elastic band 11 is a continuous singular band having a catheter retaining pocket 12 positioned along a portion of the inside surface of the elastic band 11. The catheter retaining pocket 12 is shown in dotted lines.

FIG. 2 depicts a front plan view of a portion of the inside surface of a peritoneal dialysis security band 10 showing the catheter retaining pocket 12.

FIG. 3 depicts a front view of a peritoneal dialysis security band 10 positioned about the waist of an individual. The catheter retaining pocket 12 is depicted as dotted lines and is positioned along the inside surface of the elastic band 11. A catheter is shown in dotted lines and is positioned within the catheter retaining pocket 12.

FIG. 4 depicts a front plan view of a peritoneal dialysis security band 10 positioned about the hips or waist of an individual with the elastic band 11 folded forward to reveal a catheter partially positioned in the catheter retaining pocket 12.

### DETAILED DESCRIPTION

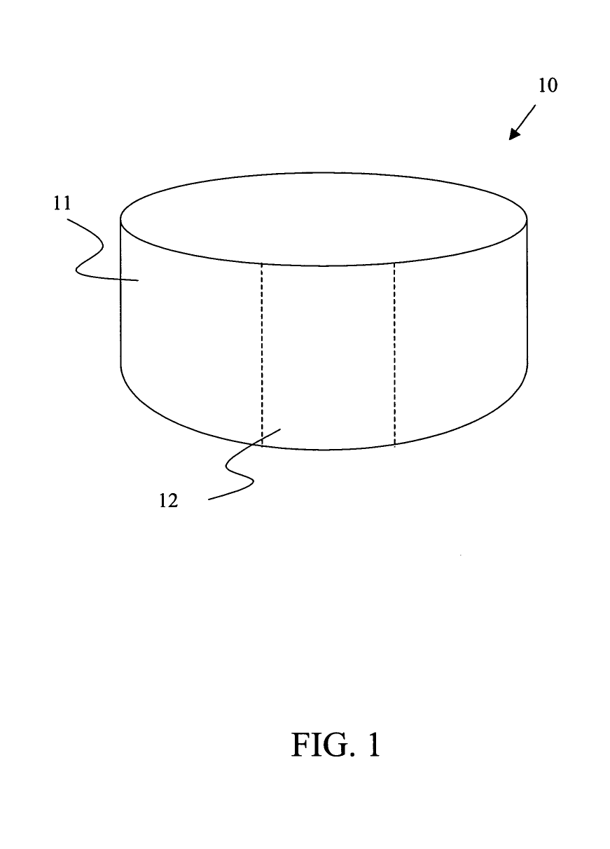

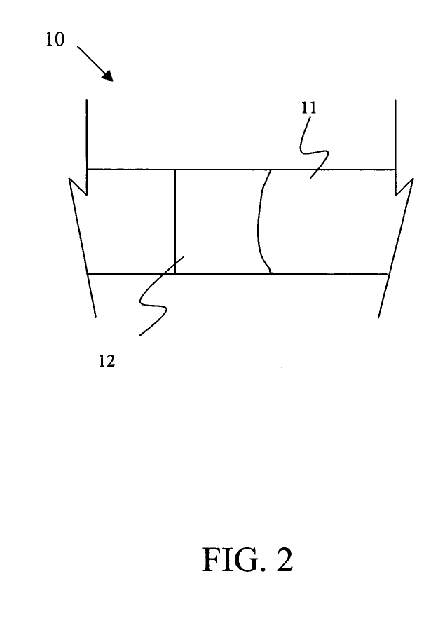

Referring to FIG. 1 and FIG. 2, the disclosed dialysis security band 10 includes an elastic band 11 and a catheter retaining pocket 12. The catheter retaining pocket 12 is able to accept the external portion of the catheter and is located along a portion of the elastic band 11. Preferably the catheter retaining pocket 12 is positioned along a portion of the inside surface of the elastic band 11 however the catheter retaining pocket 12 may be positioned along a portion of the outside surface of the elastic band 11. As will be realized, the present device provides significant advantages over previous catheter belts. For example, the dialysis security band 10 of the present invention reduces or eliminates binding, pinching, scratching, or irritation of the skin; reduces or eliminates the pulling or tugging of the catheter; and reduces or eliminates the likelihood of the garment coming undone. Among these and other benefits, the peritoneal dialysis band may provide the patient with additional back support and may be machine washable.

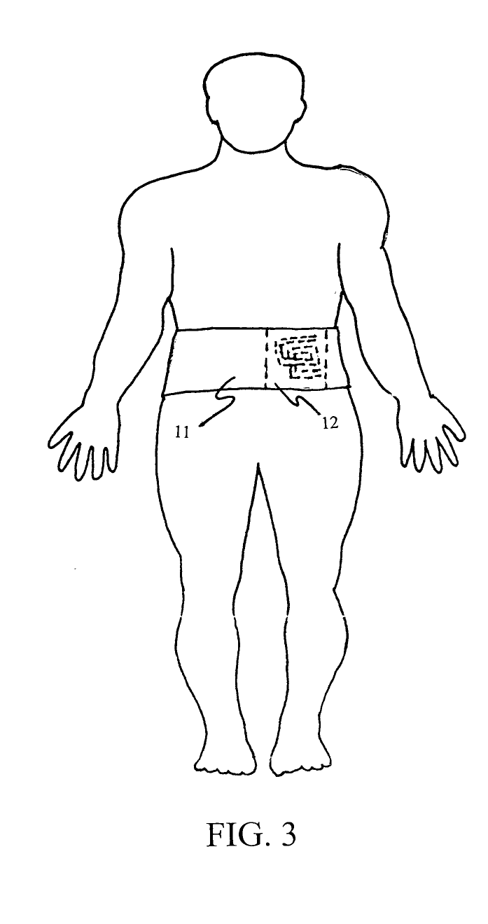

Referring to FIG. 3, the security band 10 is worn about the waist or hips. The security band 10 may be placed about the patient's waist by stretching the elastic band 11, inserting the patient's legs through the elastic band 11, sliding the security band 10 upwards along and about the patient's legs ending at about the waist or hips of the patient and releasing the elastic band 11. The catheter retaining pocket 12 may be exposed for insertion of the catheter by stretching the elastic band 11 generally outward or folding the elastic band 11 generally forward. Therefore security band 10 should be aligned such that the catheter retaining pocket 12 is oriented near the exterior portion of the catheter.

The peritoneal security band 10 may be offered in a variety of sizes to accommodate a variety of waistlines or may be offered in a one-size-fits-all configuration. The size may generally correspond to the size of the elastic band 11. As a non-limiting example the peritoneal dialysis security band 10 may be provided in extra small (XS), small (S), medium (M) and large (L) sizes. More specifically, an extra small may be offered to accommodate a waist size from about sixteen inches to about twenty-six inches, a small from about twenty-six inches to about thirty-six inches, a medium from about thirty-six inches to about forty six inches, and a large from about forty-six inches to about fifty-six inches. Alternatively, a one-size-fits-all security band 10 may accommodate a waist size from about sixteen to about fifty-six inches. However the present invention encompasses larger and smaller sizes.

The elastic band 11 is nondetachable and is worn generally about waist or hips of the patient such that the catheter may be retained in the catheter retaining pocket 12. The elastic band 11 may be provided in a variety of sizes allowing the peritoneal dialysis security band 10 to fit a variety of waistlines. The vertical width of the elastic band 11 may be variable and should be sufficiently wide to facilitate a catheter retaining pocket 12. Preferably the vertical width is from about three inches to about six inches wide.

The elastic band 11 may be constructed entirely of an elastic material or may be constructed in part from an elastic material and in part from an inelastic material. The elastic material allows the peritoneal dialysis security belt to stretch and accommodate a variety of waistlines. Examples of appropriate elastic materials or fabrics include those used in the garment industry such as but not limited to spandex, lycra and elastic rubber. In addition, the elastic band 11 may utilize a variety of inelastic materials or fabrics used in the garment industry such as but not limited to cotton, cotton blend, nylon, polyester, and inelastic rubber. Materials may be used alone or in combination with one another and are preferably machine washable and breathable.

In one configuration, the elastic band 11 is constructed entirely from a machine washable elastic fabric. The elastic fabric is cut to the desired size and the opposing ends are sewn together or irreversibly affixed to form a non-detachable elastic band.

In another configuration, the elastic band 11 is constructed from two or more elastic portions in vertical alignment with at least one inelastic portion. The inelastic portion may function to attach the elastic portions together and may limit the amount that the elastic band 11 may stretch. For example, an elastic rubber portion may extend horizontally and be vertically aligned with one or more additional elastic rubber portions. An inelastic polyester portion may be vertically aligned between and attached to the elastic rubber portions. There may exist spaces or gaps within the inelastic polyester portion where no material or fabric exists thereby allowing the elastic portion to stretch horizontally.

In another configuration, the elastic band 11 is constructed from an elastic material in horizontal alignment with an inelastic material creating a region that stretches horizontally and a region that does not substantially stretch horizontally. In this configuration distinct panels may be provided that either stretch or do not stretch and therefore control the adjustment size of the security band. For example, the opposing ends of a lycra panel may be sewn to a panel of a cotton blend. In this example the lycra allows the elastic band 11 to stretch and therefore adjust to a larger waistline while the cotton blend limits the amount of stretching of the elastic band thereby limiting the adjustment of the elastic band 11.

In another configuration, the elastic belt 11 is constructed in part from an elastic material and in part from an inelastic material where the materials are combined in a single panel or region. For example, an elastic rubber may be encased, surrounded or interweaved with a polyester fabric. In this example the polyester fabric may be bunched up until the elastic rubber is stretched. At this point the polyester fabric becomes taught and the elastic band 11 has reached its limit as to size.

Referring to FIG. 3, the catheter retaining pocket 12 functions to retain the catheter to the elastic band 11. The catheter retaining pocket 12 is positioned along a portion of the inside of the elastic band 11. The catheter retaining pocket 12 should be sufficiently large that the external portion of the catheter may be placed substantially within the catheter retaining pocket 12. Preferably, the catheter retaining pocket 12 is about 4 inches horizontally long by about 6 inches vertically wide however any dimensions allowing the retention of a catheter are sufficient.

The catheter retaining pocket 12 may be constructed from a variety of elastic and inelastic materials such as spandex, lycra, elastic rubber, cotton, cotton blend, nylon, polyester, and inelastic rubber or any combination thereof. Preferably the catheter retaining pocket 12 is constructed from at least in part an elastic material to further assist the retention of the catheter.

The catheter retaining pocket 12 may be formed separately then affixed to the elastic band 11. Alternatively the catheter retaining pocket 12 may be formed upon sewing or affixing an elastic or inelastic material or fabric to the elastic band 11 resulting in a pocket or pouch able to accept a catheter. As another non-limiting example, the catheter retaining pocket 12 may be formed by overlapping the ends of the elastic band 11 and sewing along three of four perimeter sides of the overlapping portion thereby forming a pocket able to accept a catheter.

The present invention also includes a method of retaining the external portion of an implanted catheter including placing the peritoneal dialysis security band 10 of the present invention about the waist or hips of an individual and inserting the external portion of the implanted catheter in the catheter retaining pocket 12. The catheter may be placed in the catheter retaining pocket 12 after positioning the elastic band 11 about the waist or hips.

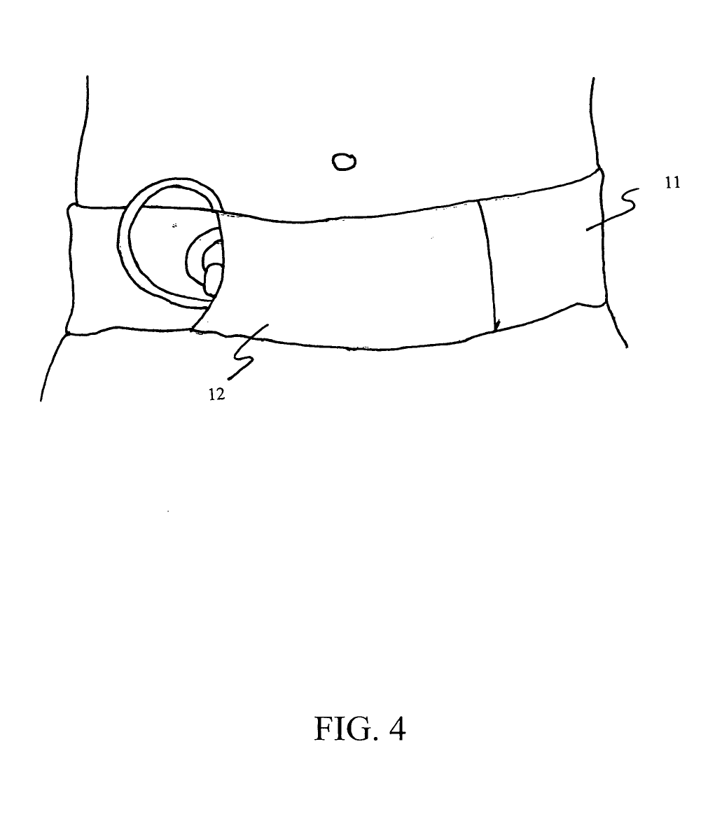

Once the patient must perform the dialysis procedure, the catheter may be easily accessed. The elastic band may be stretched generally outward or folded generally forward as shown in FIG. 4, allowing access to the catheter retaining pocket 12. The catheter may then be removed from the catheter retaining pocket 12 and used according to the physician's instructions. Likewise, reinsertion of the catheter into the catheter retaining pocket 12 may involve stretching the elastic band 11 generally outward or folding the elastic band 11 generally forward exposing the catheter retaining pocket 12 and reinserting the catheter into the catheter retaining pocket 12.

All publications including patent documents referred to in this application are incorporated by reference in their entirety for all purposes to the same extent as if each individual publication were individually incorporated by reference.

All headings are for the convenience of the reader and should not be used to limit the meaning of the text that follows the heading, unless so specified.

1. A peritoneal dialysis security band comprising:
a) an elastic band, wherein said elastic band is not detachable;
b) a catheter retaining pocket positioned along a portion of said elastic band, wherein said pocket is able to accept a catheter.

2. The peritoneal dialysis security band according to claim 1, wherein said elastic band comprises an inner surface and an outer surface, further wherein said catheter retaining portion is positioned along a portion of said inner surface.

3. The peritoneal dialysis security band of claim 1, wherein said elastic band is constructed at least in part from an elastic material selected from the group consisting of spandex, lycra, and elastic rubber.

4. The peritoneal dialysis security band according to claim 1, wherein said elastic band is constructed in part from an inelastic material.

5. The peritoneal dialysis security band according to claim 4, wherein said inelastic material is selected from the group consisting of cotton, nylon, polyester and inelastic rubber.

6. The peritoneal dialysis security band according to claim 1, wherein said elastic band is foldable material.

7. The peritoneal dialysis security band according to claim 1, wherein said elastic band is constructed from a machine washable material.

8. The peritoneal dialysis security band according to claim 1, wherein said elastic band has a width from about 3 inches to about 6 inches.

9. The peritoneal dialysis security band according to claim 1, wherein said catheter retaining pocket is constructed at least in part from an elastic material or an inelastic material.

10. A method of retaining an external portion of an implanted dialysis catheter comprising:
a) placing the peritoneal dialysis security band according to claim 1 about the waist of an individual;
b) inserting the external portion of a peritoneal dialysis catheter in said catheter retaining pocket.

11. The method according to claim 10, wherein said peritioneal dialysis security band is positioned generally about the waist prior to inserting the catheter in the catheter retaining pocket.
